(19) United States
(12) Patent Application Publication
(10) Pub. No.: US 2007/0292531 A1
(43) Pub. Date: Dec. 20, 2007

(54) COMPOSITION FOR ORAL USE AND METHODS FOR APPLICATION OF SAME

(76) Inventors: Tim Clarot, Phoenix, AZ (US); Regina Miskewitz, Phoenix, AZ (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/748,668

(22) Filed: May 15, 2007

Related U.S. Application Data
(60) Provisional application No. 60/800,639, filed on May 15, 2006.
(60) Provisional application No. 60/800,637, filed on May 15, 2006.

(51) Int. Cl. A61K 31/44; A61K 33/30; A61K 36/54; A61K 36/61; A61Q 11/00

(52) U.S. Cl. 424/643; 424/739; 424/742; 514/358

(57) ABSTRACT

A bactericidal composition for improving oral health and a method of using and forming the composition are disclosed. The composition includes one or more active ingredients and a carrier configured to maintain the composition in contact with a surface for an extended period of time to promote oral health and hygiene.

### CROSS REFERENCE TO RELATED APPLICATIONS

This application claims the benefit of U.S. Patent Application Ser. No. 60/800,639, entitled ORAL CARE PROTECTION SYSTEM, filed May 15, 2006 and U.S. Patent Application Ser. No. 60/800,637, entitled BACTERIALCIDAL COMPOSITION FOR ORAL USE AND METHODS FOR APPLICATION OF SAME, filed May 15, 2006.

### FIELD OF INVENTION

The present invention generally relates to compositions for promoting oral health, and more particularly to compositions for neutralizing and/or eliminating bacteria in the mouth and on teeth, gums and other oral components, and for promoting oral tissue health and support.

### BACKGROUND OF THE INVENTION

Various oral microorganisms, such as bacteria are commonplace in the mouth and on the oral components of various mammals. For example, there are about 400 to about 600 different species of microorganisms found in a human mouth. While some level of bacteria and other microorganisms is not necessarily harmful, if increased levels of microorganisms and/or a shift of balance of the microorganisms occurs, significant problems may develop. For example, the increase in number or shift in balance may contribute to plaque and tartar formation, tooth decay, gingivitis, periodontitis (which may lead to tooth loss and other issues), and other health problems.

The plaque formed can be defined as an organized, coherent, gel-like or mucoid material that includes microorganisms in an organic matrix derived from saliva and extracellular bacterial products such as glucans, fructans, enzymes, toxins, and acids.

Plaque may also contain other cells, such as desquamated epithelial cells, and inorganic components, such as calcium and phosphate. Generally transparent and sticky, plaque attaches to hard surfaces such as teeth and dental devices. Once plaque forms on a surface, the plaque can usually be removed by brushing and flossing the affected areas. If not removed, however, the presence of plaque can give rise to further increased levels of oral bacteria which may further contribute to tartar formation (which forms from calcified plaque), tooth decay, gingivitis, periodontitis, and other health problems.

The tartar formed is a hard, calcified plaque material that exhibits a yellowish or brownish color. Tartar forms as a result of minerals (e.g., those present in saliva and gum pockets) reacting with plaque material to form a rough calculus. The calculus can form on surfaces not covered by the oral mucosa (supragingival) or on surface located apical to the soft tissue margin of the gingiva (subgingival).

If left untreated, tartar buildup can be problematic in several regards. For example, the rough, porous surface of tartar serves as a breeding ground for additional bacteria, which can calcify and form additional tartar, and again the bacteria growth can, in turn, lead to tooth decay, gum disease, tooth loss, as well as systemic health problems.

In addition to the health concerns, tartar is a cosmetic problem due to its yellowish or brownish color, which stains teeth and dental devices. Further still, because the surface of tartar is rough and porous, the tartar absorbs colors from other sources (e.g., coffee, tea, smoke, and the like), and thus the presence of tartar exacerbates cosmetic tooth coloration typically associated with such other sources. Further still, bacteria may result in halitosis—known otherwise as “bad breath.”

Typical methods of neutralizing or eliminating bacterial and plaque and preventing tartar buildup include brushing with toothpaste, containing tartar crystal inhibiting agents such as pyrophosphates and using antibacterial mouthwash. Although such toothpastes and mouthwashes, if used regularly, may help prevent additional buildup of tartar and plaque, they are not always that effective at eliminating or neutralizing bacteria between uses.

Saliva is the fluid produced by the salivary glands, which are situated around the jaw and under the tongue. Saliva naturally aids in the prevention of the deposition of plaque and the like by keeping the mouth clean and helps to prevent infection. It protects teeth, and a lack of saliva can increase the risk of developing tooth decay or gum disease.

However, some people experience periodic decreases in saliva production, particularly during the night time. This is because, while sleeping, the production of saliva slows down, causing sticky plaque and odor-causing bacteria to build up and penetrate oral surfaces, creating the feeling of “nighttime mouth.” That is, for a normal person there is a significant reduction in saliva during rest or sleep.

Accordingly, oral compositions for protecting teeth, gums and other oral components, particularly prior to extended periods of sleep or rest are desired.

### SUMMARY OF THE INVENTION

The present invention provides a composition for improving oral health. The composition works by eliminating and/or neutralizing microorganisms, such as bacteria in the mouth on teeth, gums and other oral components. Preliminarily, as used herein, “eliminating” and “neutralizing” are generally used to describe a reduction, either partial or complete, in oral microorganisms, and the use of one term should not be read as precluding the other. In this regard, the present invention assists in neutralizing microorganisms and preventing and/or mitigating plaque build-up, tartar build-up and gingivitis, as well as reducing the effects of the same.

Oral compositions applied in accordance with the present invention may provide the foregoing benefits, particularly when applied prior to extended periods of sleep or rest, when teeth, gums and the like are more susceptible to the afflictions noted above. In this case, the composition is configured to remain in contact with a surface for an extended period of time.

While the ways in which the invention addresses the various drawbacks of known compositions and methods will be described in more detail below, in general, the present invention provides a relatively inexpensive, safe composition for the neutralization of oral bacteria and other microorganisms. Compositions of the present invention and methods of using the compositions are relatively easy to use, do not require a visit to a dentist office, and do not damage the surface of enamel.

In accordance with one embodiment of the invention, a bactericidal composition includes a base and one or more active ingredients for neutralizing bacteria and other microorganisms and preventing the disposition of plaque, reducing tartar build-up and/or removing tartar from a surface, and a viscous carrier designed to maintain the active ingredient(s) in contact with the surface for an extended period of time. Additionally, benefits from bacteria reduction included oral health, tissue support, and/or tissue regeneration. Such support and regeneration may result in a decrease of bleeding at the gumline.

Exemplary compositions in accordance with the present invention have a viscosity greater than about 20,000 cp, preferably greater than 30,000 cp, and more preferably greater than about 35,000 cp. The viscosity of the compositions may range from about 20,000 to about 250,000 cp, preferably about 25,000 to about 100,000, and more preferably about 30,000 to about 50,000 cp. Suitable active ingredients include cetylpyridinium chloride (CPC), zinc salts, other antimicrobial agents, and other ingredients known to reduce tartar and/or the formation of plaque.

In accordance with another embodiment of the invention, a bactericidal composition includes a plurality of ingredients to neutralize bacteria and other microorganisms and a carrier that includes a thickening agent. The bactericidal composition is configured to maintain the plurality of active ingredients in contact with a surface for an extended period of time. Exemplary thickening agents include hydroxyethylcellulose, glycerin, and other pharmaceutically acceptable thickeners, and exemplary active ingredients include CPC, zinc salts, other antimicrobial agents, and similar agents known to reduce tartar or plaque.

In accordance with various embodiments of the invention, a method of applying a bactericidal composition includes applying a composition having a viscosity greater than about 20,000 cp directly to the surface. In accordance with various aspects of this embodiment, the composition is dispensed from a sealed container by rupturing the sealed container and applying a single does of the composition directly to the surface.

### BRIEF DESCRIPTION OF THE DRAWINGS

The exemplary embodiments of the present invention will be described in connection with the appended drawing figures in which like numerals denote like elements and:

FIG. 1 illustrates a perspective view of a dispensing device in accordance with exemplary embodiments of the invention; and

FIGS. 2A-2D illustrate, respectively, a left view, a rear view, a right view and a top view of the dispensing device illustrated in FIG. 1.

### DETAILED DESCRIPTION

As noted above, the present invention provides a bactericidal composition that eliminates and/or neutralizes oral bacteria and other microorganisms for the, gums and other oral structures, thus preventing and/or reducing tartar and plaque buildup and related maladies and provides for tissue support and regeneration of gums and other oral components, and a method of using the composition. The present invention may also protect teeth, gums and other oral structures, particularly during sleep and/or periods of rest, thus preventing and/or reducing tartar and plaque buildup, gingivitis, and the like. The compositions and methods of the present invention can be used for various animals, and are particularly well suited for the treatment humans.

As used throughout this application, the term surface includes any surface on which bacteria, plaque or tartar may reside or form. Exemplary surfaces include teeth (both supragingival and subgingival) and dental devices such as bridges, crowns, fillings, braces, and the like.

In accordance with various embodiments of the invention, a bactericidal composition for affecting the benefits set forth above includes at least one active ingredient and a base or “carrier.” As explained in greater detail below, a bactericidal composition is designed to maintain contact with a surface, preferably for an extended period of time, to allow both the active ingredient(s) to stay in contact with areas on the surface susceptible and known to have bacterial/microbiological build-up and to prevent or reduce the effects of the same.

In accordance with various aspects of the present invention, exemplary active ingredients include one or more of the following: cetylpyridinium chloride (CPC), dicalcium phosphate dehydrate, hydrogen peroxide, sanguinaria extract, sodium bicarbonate, sodium lauryl sulfate, stannous fluoride, sodium fluoride, sodium MFP (sodium monofluorophosphate), zinc salts such as zinc chloride, zinc acetate, zinc citrate, zinc oxide, and zinc gluconate, alkyl dimethyl amine oxide, alkyl dimethyl glycine, eucalyptol, menthol, methyl salicylate, thymol, sodium citrate, peppermint oil, sage oil, polymethylsiloxane, polxamer, and stannous pyrophosphate.

An amount of the active ingredient suitable for use with a bactericidal or antibacterial composition varies in accordance with the dosage size and particular ingredient. In accordance with particular examples, the active ingredient is present in an amount of about 0.001 wt % to about 1.5 wt %, preferably about 0.025 wt % to about 1.0 wt %, and more preferably, about 0.05 wt % to about 0.7 wt %. All percentages set forth herein are in weight percent of the total composition, unless otherwise indicated. An amount of active may also desirably be selected to be within a range recommended by a regulatory agency, such as the Food and Drug Administration of the USA.

In accordance with various aspects of this exemplary embodiment of the invention, the active ingredient includes CPC. In this case, the CPC is present in an amount of about 0.001% to about 1%, preferably about 0.01% to about 0.5%, and more preferably about 0.05% to about 0.25% or 0.045% to about 0.1%.

In accordance with other embodiments of the present invention, the active ingredient includes zinc gluconate. In this case, the zinc gluconate is present in an amount of about 0.001% to about 1.5%, preferably about 0.01% to about 1.0%, and more preferably about 0.05% to about 0.75%.

In accordance with various exemplary embodiments of the present invention, a bactericidal or antibacterial composition is configured to adhere to oral components for extended period of time. For example, bactericidal compositions may include a thickener to obtain a desired viscosity such as the exemplary viscosities noted herein.

Suitable thickening agents include substances which increases the viscosity of the composition, cause the composition to gel or coagulate, or the like, such as food-grade or pharmaceutical grade thickeners, including, for example, hydroxyethylcellulose, glycerin, carrageenan, guar gum, methylcellulose, methyethylecellulose, aloe vera, and the like. The thickener may be present in an amount of about 0.01 to about 10%, preferably about 0.1 to about 7%, and more preferably about 1% to about 5% or about 0.5% to about 3%.

In various embodiments, a bactericidal composition may also include a humectant such a glycerin, which may be present in an amount of about 0.01% to about 15%, preferably about 0.1% to about 10%, and more preferably about 1 to about 7%. When used, the humectant may facilitate maintaining a bactericidal composition in a liquid form and may help maintain a desired viscosity of the composition. In addition, glycerin may facilitate maintaining one or more of the active ingredients in an ionic form and may facilitate the transport of the active ingredients through a bactericidal composition.

A bactericidal composition may also include a suitable diluent. Exemplary diluents suitable for use with the present a bactericidal composition include Sorbitol, Xylitol, Mannitol, water, alcohols, and oils. In accordance with particular examples of the invention, a bactericidal composition includes purified water in an amount of about 80% to about 99%, preferably about 85% to about 95%, and more preferably about 88% to about 92%.

A bactericidal composition may also include sugar alcohols such as sorbitol that act as a sweetener and also as a humectant and/or emulsifier. When used, sorbitol or other sugar alcohol is present in an amount of about 0.001% to about 0.5%, preferably about 0.01% to about 0.1%, and more preferably about 0.025% to about 0.075%. However, higher concentrations are also within the scope of this invention.

A bactericidal composition may also include a natural or artificial sweetener such as sucralose, sodium saccharin, Acesulfame K, aspartame, and the like, which, when included in a bactericidal composition, is present in an amount of about 0.001% to about 1.5%, preferably about 0.01% to about 1%, and more preferably about 0.25% to about 0.75%.

Optionally, colorants may also be added to bactericidal compositions as desired. In accordance with various aspects, a bactericidal composition includes colorants, such that when a bactericidal composition is applied to or proximate the gingiva, the composition has a color indicative of healthy gingiva—i.e., the composition is pink in color. Providing a bactericidal composition having a color indicative of healthy gingiva provides added incentive to users to continue using a bactericidal composition.

Such colorants may be present in any desired amount. The colorants may include Red #33 and Red #40, available from Pylam in an effective amount. For example, the colorants may include Red #33 and/or Red #40, available from Pylam, in an amount of about 0.000005% to about 1%, preferably about 0.00050% to about 0.5%, and more preferably about 0.001% to about 0.1%.

Additionally and/or alternatively, colorants may be added to indicate a flavor. For example, green and blue colorants can be used to indicate mint flavors, and reds can be used to indicated cinnamon and the like. In accordance with some examples, any suitably approved colorant may be used. Examples include FD&C Blue #1, D&C Green #5, FD&C Yellow #5, and FD&C Yellow #6.

The composition also optionally includes flavorants such as cinnamon bark oil, clove bud oil, mint flavorings, citrus flavorings, and the like. By way of one particular example, a composition includes cinnamon bark oil present in an about of about 0.001% to about 2%, preferably about 0.01% to about 1%, and more preferably about 0.1% to about 0.5%; and clove bud oil in an amount of about 0.001% to about 2%, preferably about 0.002% to about 1%, and more preferably about 0.003% to about 0.0075%.

The composition is configured to maintain the composition in contact with a surface for an extended period of time. For example, because the composition is viscous and “adheres” to a surface to which it is applied, relatively small amounts of the composition and consequently the active agent(s), can be used.

This is advantageous, not only from an economic viewpoint, but also because the composition may ultimately be ingested, and, in this case, reduced amounts of the composition and the active ingredients may be desirable. Additionally, the relatively high viscosity allows for relatively select placement of the composition on a surface. For example, as described in more detail below, the composition can be selectively applied to or proximate portions of, for example, a tooth and/or a gumline.

Exemplary compositions have a viscosity greater than about 20,000 cp, preferably greater than 30,000 cp, and more preferably greater than about 35,000 cp. By way of more particular examples, the viscosity of compositions range from about 20,000 to about 250,000 cp, preferably about 25,000 to about 100,000, and more preferably about 30,000 to about 50,000 cp, and yet more preferably about 35,000 to about 45,000 cp. The viscosity values as set forth herein are measured using a Brookfield, Model DV-II+ Pro viscometer using spindle #6, 10 RPM for 90 seconds at 25 C.

Multiple Active Ingredients

In accordance with another embodiment of the invention, a bactericidal composition includes multiple active ingredients in a carrier, which includes a thickening agent. In this case, a bactericidal composition includes a plurality of any of the active ingredients noted above and any thickening agents noted above in the weight percents noted above. A bactericidal composition may also include any of the optional ingredients such as sweeteners, flavorants, and colorants as set forth herein.

By way of example, a bactericidal composition in accordance with this embodiment of the invention includes CPC and zinc gluconate as the active ingredients. In one case, the CPC is present in an amount of about 0.001% to about 1%, preferably about 0.01% to about 0.5%, and more preferably about 0.05% to about 0.25%; and the zinc gluconate is present in an amount of about 0.001% to about 1.5%, preferably about 0.01% to about 1.0%, and more preferably about 0.05% to about 0.75%.

Active Ingredient and Colorant

As noted above, bactericidal compositions in accordance with various embodiments of the invention include one or more active ingredients and a colorant indicative of healthy gingiva. In one case, the color of a bactericidal composition is more than merely decorative. It serves the function of encouraging those that use the product to continue to use the product because there is an immediate appearance, upon application of the composition, the composition is promoting healthy gingiva.

### SPECIFIC EXAMPLES

The following non-limiting examples illustrate an exemplary composition in accordance with various embodiments of the invention. These examples are merely illustrative, and it is not intended that the invention be limited to the example. Compositions in accordance with the present invention may include the ingredients listed below as well as additional and/or alternative inert materials, preservatives, and other constituents typically found in compositions for removing and/or preventing tartar buildup. In the case where exemplary inert materials and/or preservatives are listed, these ingredients are merely exemplary, and it is understood that other similar ingredients may be substituted for the materials listed in the examples below.

### Example 1

### CPC and Zinc Gluconate

A pale light pink viscous gel, having a viscosity of about 40,000 cp, with cinnamon-clove characteristic odor and taste is formed by admixing the following ingredients in the amounts shown.

TABLE 1

| | | | Exemplary |
|---|---|---|---|
| Ingredient | Supplier | Weight % | Wt % Range |
| Purified Water | Copacker | 91.504 | 80-99 |
| Glycerin USP | Acme-Hardesty | 5.000 | 0.01-15 |
| HEC 250 HX | Hercules-Aqualon | 2.000 | 0.01-10 |
| Sorbitol | Roquette | 0.050 | 0.001-0.5 |
| Sucralose | Tate & Lyle | 0.400 | 0.001-1.5 |
| Cetylpyridinium | Dishman | 0.100 | 0.001-1 |
| Chloride | Pharmaceuticals | | |
| Zinc Gluconate | American | 0.592 | 0.001-1.5 |
| USP | International | | |
| Cinnamon Bark | Spectrum | 0.250 | 0.001-2 |
| (Oil) | | | |
| Clove Bud (Oil) | Spectrum | 0.005 | 0.001-2 |
| Red #40 (1% sol.) | Pylam | 0.099 | 0.000005-1 |

A clinical study comparing the composition of Example 1 to a placebo and a mouth rinse including CPC was conducted. Each group started with 25 people each. Prior to a three-week evaluation, one person from the Example 1 group and one person from the mouth rinse group dropped out. At the conclusion of a three-week test period, there was an observable difference in the quality and thickness of calculus (tartar) in the group using the composition of Example 1, compared to the placebo and the mouth rinse. A significant disruption of calculus bridges or masses of calculus was observed in subjects using the composition of Example 1. A softening of the calculus was also observed for those using the composition of Example 1.

### Example 2

In a further example, a clinical study comparing the bactericidal composition of Example 1 below to a placebo and a mouth rinse including CPC was conducted. Each group started with 25 people each. Prior to a three-week evaluation, one person from the Example 1 group and one person from the mouth rinse group dropped out. At the conclusion of a six (6) week test period, there was an observable/measurable difference in the improvement of the glum bleeding index (GBI) for the group using the composition of Example 1, compared to the groups using the placebo and the rinse.

### Example 3

Bacteriacidal compositions of the present invention also provide for the neutralization of bacteria, thus promoting oral health, for example, by reducing plaque and tartar build-up, bad breath and gingivitis. Additionally, the neutralization of bacteria may provide for tissue (e.g., gums) support and regeneration, as well as reduced bleeding. Subjects that used the bactericidal composition in accordance with the present invention over a six week period showed tissue regeneration and health, calculus reduction and a reduction of blood spots

Method of Using Bactericidal Composition

A method of providing bactericidal composition to oral surfaces includes applying the composition including one or more of the active ingredients described herein to the surface and maintaining contact of the bactericidal composition with the surface for an extended period of time, for example, prior to going to bed, when the oral components noted above have a higher vulnerability to the afflictions noted above.

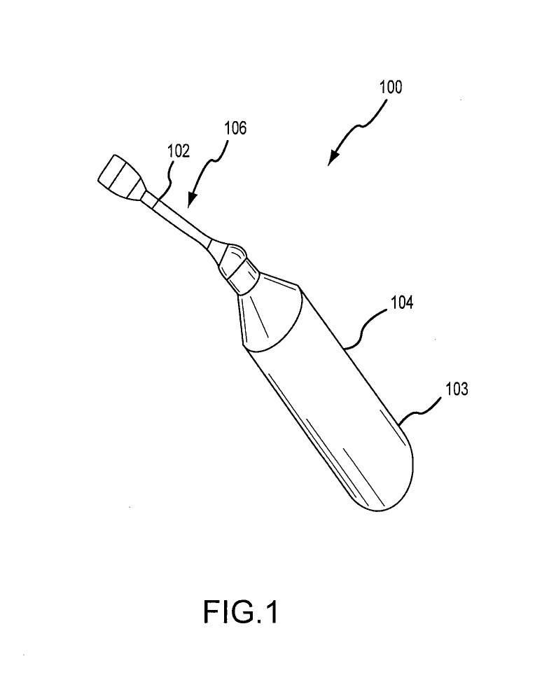

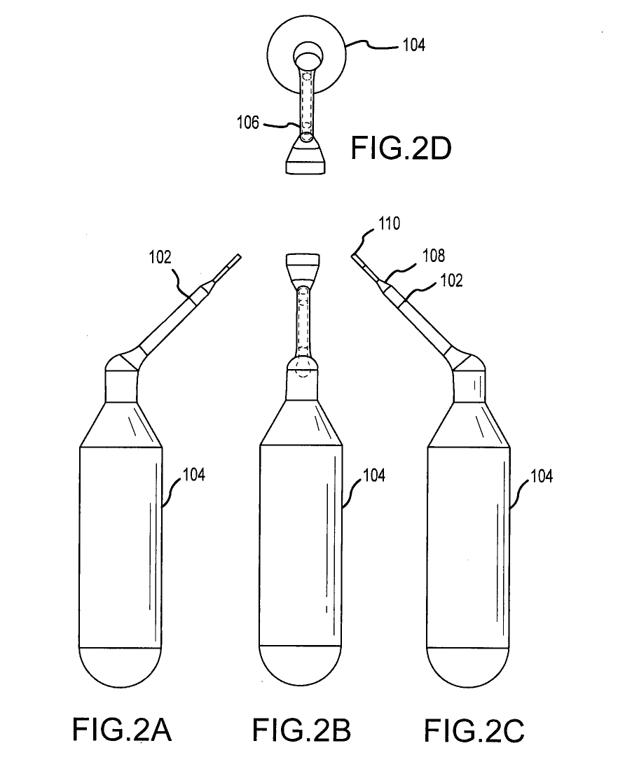

A bactericidal composition may be applied using a variety of methods such as with a swab, a syringe, or using any other means capable of dispensing the composition. FIG. 1 and FIGS. 2A-2C illustrate a single dose dispensing device 100 suitable for applying a bactericidal composition to a surface. A bactericidal composition is dispensed from device 100 by severing or rupturing a portion of device 100 (e.g., at or near area 102 that has been scared) to expose composition 104 contained within device 100. As noted above, a dose size may vary in accordance with several factors. Exemplary dose sizes for purpose of illustrate range from about 0.3 mg to about 8.0 mg, preferably about 1 mg to about 7 mg, and more preferably about 2 mg to about 5 mg.

In accordance with various aspects of this embodiment, a bactericidal composition is selectively applied though chute 106 to a tooth/gingiva interface, such that it contacts both supragingival and subgingival areas where oral bacteria often occurs (and where plaque build-up and tartar is likely to form) thereby neutralizing bacteria in those areas.

Finally, although exemplary embodiments of the present invention are set forth herein, it should be appreciated that the invention is not so limited. Various modifications, variations, and enhancements in composition and method set forth herein may be made without departing from the spirit and scope of the present invention.

What is claimed is:

1. A bactericidal composition for oral application, the composition comprising:
a carrier comprising a thickener and a diluent;
cetylpyridinium chloride; and
a zinc salt.

2. The bactericidal composition of claim 1, wherein the composition has a viscosity greater than about 20,000 centipoise.

3. The bactericidal composition of claim 1, wherein a viscosity of the composition is greater than about 30,000 centipoise.

4. The bactericidal composition of claim 1, wherein a viscosity of the composition is greater than about 35,000 centipoise.

5. The bactericidal composition of claim 1, further comprising a humectant.

6. The bactericidal composition of claim 1, further comprising a sugar alcohol.

7. The bactericidal composition of claim 1, further comprising a colorant indicative of healthy gingiva.

8. The bactericidal composition of claim 1, wherein the viscosity of the composition is about 25,000 cp to about 100,000 cp.

9. The bactericidal composition of claim 1, wherein the viscosity of the composition is about 30,000 cp to about 50,000 cp.

10. A bactericidal composition, the composition comprising:
about 0.001 weight percent to about 1 weight percent bactericidal agent;
about 0.001 weight percent to about 1.5 weight percent zinc salt;
about 80 weight percent to about 99 weight percent diluent; and
about 0.01 weight percent to about 10 weight percent thickener.

11. The bactericidal composition according to claim 10, wherein a viscosity of the composition is greater than about 20,000 centipoise.

12. The bactericidal composition according to claim 10, wherein a viscosity of the composition is greater than about 30,000 centipoise.

13. The bactericidal composition according to claim 10, wherein a viscosity of the composition is greater than about 35,000 centipoise.

14. The bactericidal composition according to claim 10, further comprising a humectant.

15. The bactericidal composition according to claim 10, further comprising a sugar alcohol.

16. The bactericidal composition according to claim 10, further comprising a colorant indicative of healthy gingiva.

17. The bactericidal composition according to claim 10, wherein the viscosity of the composition is about 25,000 cp to about 100,000 cp.

18. The bactericidal composition according to claim 10, further comprising an essential oil.

19. The bactericidal composition according to claim 10, wherein the essential oil comprises cinnamon bark oil.

20. The bactericidal composition according to claim 10, wherein the essential oil comprises clove bud oil.
